(19) United States
(12) Patent Application Publication
(10) Pub. No.: US 2019/0344013 A1
(43) Pub. Date: Nov. 14, 2019

(54) SYSTEM, AN INFUSION DEVICE FOR INFUSING A SOLUTION AND A METHOD THEREOF

(71) Applicant: Sanket PISAT, Mumbai (IN)

(72) Inventor: Sanket PISAT, Mumbai (IN)

(21) Appl. No.: 16/470,582

(22) PCT Filed: Jun. 23, 2017
(86) PCT No.: PCT/IN2017/050259

§ 371 Date: Jun. 18, 2019

(30) Foreign Application Priority Data
Jan. 17, 2017 (IN) 201721001762

(51) Int. Cl. A61M 5/158; A61M 5/32

(52) U.S. Cl. CPC A61M 5/158; A61B 17/3478; A61M 2005/1588; A61M 5/329

(57) ABSTRACT

An infusion device includes a hollow needle with a tube that confirms puncturing of blood vessel by observing blood through the tube, as a result of which the hollow needle is withdrawn from the blood vessel and repositioned on the visceral organ. This repositioning continues till no blood is observed in the tube. Thereafter the vasopressin is infused by the infusion device into the tissue mass, thus ensuring that the solution like vasopressin is infused into the fibroid and not directly into the blood vessel.

### FIELD OF INVENTION

Present invention relates to a system, an infusion device for infusing a solution and a method thereof, and more particularly relates to the system, an infusion device for infusing a solution and a method thereof having a needle and a tube for observing blood and for infusion of solution such as vasopressin or the like into a tissue mass of a visceral organ and not infusing such solution directly into the blood vessel to reduce blood loss during surgeries like myomectomy or the like, and to avoid cardiovascular complications.

### BACKGROUND

Laparoscopic surgeries have been used since many years for problems associated with cysts, adhesions, fibroids, infection or the like. These surgeries are performed through 3 or 4 small holes on the skin, which includes less bleeding, less pain, faster healing, shorter hospital stay and many more. However, in surgeries for removal of fibroid or the like, which is a non cancerous growth on the uterus, also known as laparoscopic myomectomy, a solution like vasopressin or vaso constrictive agent for reducing bleeding, is injected into the fibroid before a cut is made over the uterus. The infusion of this solution like vasopressin or vaso constrictive agent constricts blood vessels to provide a clean, bloodless field for surgery and even makes the surgery easier and faster. This solution must be injected into the uterus, and not directly into a blood vessel over the uterus or fibroid. However, the blood vessels in these areas are usually very small and often not seen during surgeries, therefore, it is very likely that the solution might get injected into the blood vessel and that would result in severe blood pressure change and heart related side effect or cardiovascular complications.

U.S. Pat. No. 4,790,830 discloses an infusion needle that is substantially blunt at the distal part, so as to be capable of penetrating subcutaneous tissue and to divert blood vessels around the needle on insertion. The needle by diverting the blood vessels allows injection of toxic medicines such as lidocaine HCl through the apertures at the radial directions of the shaft that are structured near the blunt end of the needle. However, in case of laparoscopic surgeries performed on visceral organ like uterus for removal of fibroids, the blood vessels are very small or these vessels are very thin walled, therefore, it is very likely that the blunt structure of the needle of this document may not be able to divert the blood vessels and may penetrate blood vessel. Since, the blood vessels are very small, the total volume of blood aspirated into the needle would never be sufficiently large so as to create a blood column sufficiently high to stain the fluid in the syringe connected the needle red. Therefore, though the infusion needle of this document provides an option for avoiding puncturing of blood vessels by diverting them and to insert toxic medicines directly into the tissues, the needle of this document is inefficient for diverting vessels as the vessels are very small with thin walls and thus it would be very likely that the needle would puncture the small blood vessels and in such case the solution like vasopressin may directly enter the blood vessel resulting in various cardiovascular complications including heart related side effects.

Therefore, the problem to be solved is to infuse solutions like vasopressin into the fibroid or the like during laparoscopic surgeries to constrict blood vessels for making clean and bloodless field for surgery, without directly infusing the vasopressin into the blood vessels, which are very small and thin walled, and the problem is solved by providing an infusion device as in present invention that includes a hollow needle with a tube that confirms puncturing of blood vessel by observing blood through the tube, as a result of which the needle is withdrawn from the blood vessel and repositioned on the visceral organ, and this repositioning continues till no blood is observed in the tube, thereafter the vasopressin is infused by the infusion device into the tissue mass, thus ensuring that the solution like vasopressin is infused into the fibroid and not directly into the blood vessel.

### SUMMARY OF THE INVENTION

According to an embodiment of the present invention, an infusion device comprising a hollow needle having a pointing end for puncturing a tissue mass of a visceral organ, a tube at an opposite side of said pointing end of said hollow needle for observing blood in said tube when said pointing end of said hollow needle punctures a blood vessel of said visceral organ, and a longitudinal hollow cylindrical shaft connected to said tube structured to receive a solution for infusion into said tissue mass, wherein said needle is withdrawn from said blood vessel and repositioned on said visceral organ when blood is observed in said tube, and said solution is infused into said tissue mass of said visceral organ when no blood is observed in said tube after repositioning of said needle on said organ, thereby ensuring infusion of said solution into said tissue mass.

According to an embodiment of the present invention, the tissue mass is a fibroid.

According to an embodiment of the present invention, the tube is transparent to observe blood.

According to an embodiment of the present invention, the shaft is detachably connected to the tube.

According to an embodiment of the present invention, the shaft and said tube are threaded to receive each other.

According to an embodiment of the present invention, the shaft is a metallic shaft.

According to an embodiment of the present invention, the shaft includes a hub to receive a syringe carrying said solution for infusion into said tissue mass.

According to an embodiment of the present invention, the tube is substantially 1 cm long with a hollow central cylinder of substantially 0.2 cm diameter.

According to an embodiment of the present invention, the hollow needle is substantially 2.5 cm long.

According to an embodiment of the present invention, a method for infusion of a solution into a tissue mass of a visceral organ, including the steps of puncturing said visceral organ by a pointing end of a hollow needle having a tube on an opposite side of said hollow needle, observing blood in said tube of said hollow needle when said pointing end punctures a blood vessel of said visceral organ, withdrawing and repositioning said hollow needle when blood is observed in said tube of said hollow needle, and infusing said solution into said tissue mass without directly infusing into said blood vessel by a longitudinal hollow cylindrical shaft connected to said tube when blood is not observed in said tube after puncturing said tissue mass by said needle.

According to an embodiment of the present invention, the method including the step of infusing said solution into said tissue mass by infusing said solution into a fibroid.

According to an embodiment of the present invention, the method including the step of observing blood by said tube with transparent surface.

According to an embodiment of the present invention, the method including the step of infusing said solution into said tissue mass by detachably connecting said shaft with said tube of said needle.

According to an embodiment of the present invention, the method including the step of infusing said solution into said tissue mass by said shaft receiving said tube with both having threads.

According to an embodiment of the present invention, the method including the step of infusing said solution into said tissue mass by a syringe having said solution and said syringe is received by a hub of said shaft on opposite side of said shaft connected with said tube.

According to an embodiment of the present invention, the method including the step of observing blood in said tube substantially 1 cm long with a hollow central cylinder of substantially 0.2 cm diameter.

According to an embodiment of the present invention, the method including the step of puncturing said tissue mass by said pointing end of said needle substantially 2.5 cm long.

According to an embodiment of the present invention, the system for infusing a solution into a tissue mass comprising a duct with an opening on both side positioned in an incision on a body surface for forming a first passage including a flap at an inner surface of said duct in said first passage, a stick for pushing said flap to open said first passage, and an infusion device of length smaller than length of said stick forming a second passage through said infusion device to allow said stick to pass through said infusion device, said infusion device includes a hollow needle having a pointing end for puncturing said tissue mass of a visceral organ, a tube at an opposite side of said pointing end of said hollow needle for observing blood in said tube when said pointing end of said hollow needle punctures a blood vessel of said visceral organ, and a longitudinal hollow cylindrical shaft connected to said tube structured to receive a solution for infusion into said tissue mass, wherein said stick passes through said hollow needle of said infusion device when received in said infusion device for positioning said infusion device through said duct by pushing said flap by said stick, thereafter said stick is withdrawn from said infusion device, and said needle is withdrawn from said blood vessel and repositioned on said visceral organ when blood is observed in said tube, and said solution is infused into said tissue mass of said visceral organ when no blood is observed in said tube after repositioning of said needle on said visceral organ, thereby ensuring infusion of said solution into said tissue mass.

According to an embodiment of the present invention, the tissue mass is a fibroid.

According to an embodiment of the present invention, the tube is transparent to observe blood.

According to an embodiment of the present invention, the shaft is detachably connected to said tube.

According to an embodiment of the present invention, the shaft and said tube are threaded to receive each other.

According to an embodiment of the present invention, the shaft and said tube receive each other with a seal in between said shaft and said tube.

According to an embodiment of the present invention, the shaft includes a hub to receive a syringe carrying said solution for infusion into said tissue mass after withdrawing said stick from said infusion device.

According to an embodiment of the present invention, the tube is substantially 1 cm long with a hollow central cylinder of substantially 0.2 cm diameter.

According to an embodiment of the present invention, the hollow needle is substantially 2.5 cm long.

According to an embodiment of the present invention, the method for infusion of a solution into a tissue mass of a visceral organ, including the steps of positioning a duct with an opening on both side in an incision on a body surface for forming a first passage including a flap at an inner surface of said duct in said first passage, receiving a stick by an infusion device of length smaller than length of said stick forming a second passage through said infusion device to allow said stick to pass through said infusion device, positioning said infusion device through said duct by pushing said flap by said stick received by said infusion device and passing through a hollow needle of said infusion device, withdrawing said stick from said infusion device after positioning said infusion device through said duct, puncturing said visceral organ by a pointing end of said hollow needle having a tube on an opposite side of said hollow needle, observing blood in said tube of said hollow needle when said pointing end punctures a blood vessel of said visceral organ, withdrawing and repositioning said hollow needle when blood is observed in said tube of said hollow needle, and infusing said solution into said tissue mass without directly infusing into said blood vessel by a longitudinal hollow cylindrical shaft connected to said tube when blood is not observed in said tube after puncturing said tissue mass by said hollow needle.

According to an embodiment of the present invention, the method including the step of infusing said solution into said tissue mass by infusing said solution into a fibroid.

According to an embodiment of the present invention, the method including the step of observing blood by said tube with a transparent surface.

According to an embodiment of the present invention, the method including the step of infusing said solution into said tissue mass by detachably connecting said shaft with said tube of said hollow needle.

According to an embodiment of the present invention, the method including the step of infusing said solution into said tissue mass by said shaft receiving said tube with both having threads.

According to an embodiment of the present invention, the method including the step of infusing said solution into said tissue mass by a syringe having said solution and said syringe is received by a hub of said shaft on opposite side of said shaft connected with said tube.

According to an embodiment of the present invention, the method including the step of observing blood in said tube substantially 1 cm long with a hollow central cylinder of substantially 0.2 cm diameter.

According to an embodiment of the present invention, the method including the step of puncturing said tissue mass by said pointing end of said hollow needle substantially 2.5 cm long.

### BRIEF DESCRIPTION OF DRAWINGS

FIG. 1 represents an embodiment of the present invention depicting side view of an infusion device.

FIG. 2 represents an embodiment of the present invention depicting exploded view of the infusion device with a needle and a shaft.

FIG. 3 represents an embodiment of the present invention depicting the infusion device with a syringe.

FIG. 4 represents an embodiment of the present invention depicting the infusion device with a seal.

FIG. 5a represents an embodiment of the present invention depicting the infusion device and a stick outside the infusion device.

FIG. 5b represents an embodiment of the present invention depicting the infusion device and a stick inside the infusion device.

FIG. 6a represents an embodiment of the present invention depicting the infusion device and a stick outside the infusion device.

FIG. 6b represents an embodiment of the present invention depicting the infusion device and a stick inside the infusion device.

FIG. 7 represents an embodiment of the present invention depicting the infusion device with a standard 3 mm and 5 mm laparoscopic aspiration needle.

FIG. 8 represents an embodiment of the present invention depicting a graph reflecting variation in pulse rate with and without the infusion device.

FIG. 9 represents an embodiment of the present invention depicting a graph reflecting variation of systolic blood pressure with and without the infusion device.

FIG. 10 represents an embodiment of the present invention depicting a graph reflecting variation of diastolic blood pressure with and without the infusion device.

FIG. 11 represents an embodiment of the present invention depicting a bar graph reflecting comparison of difference of pulse rate, systolic blood pressure and diastolic blood pressure with and without the infusion device on study group at 1 minute.

| Description of Elements | Reference Numeral |
|---|---|
| System for infusing solution | 10 |
| Infusion device | 100 |
| Hollow Needle | 120 |
| Pointing end | 122 |
| Tube | 130 |
| Outer threads | 132 |
| Shaft | 140 |
| Inner threads | 142 |
| Hub | 144 |
| Seal | 146 |
| Stick | 148 |
| Handle | 149 |
| Syringe | 150 |
| Standard 5 mm needle | 160 |
| Standard 3 mm needle | 162 |
| Duct | 170 |
| Flap | 172 |

### DETAILED DISCLOSURE OF THE INVENTION

The embodiments of the present invention can be understood by reading following detailed description of some of the embodiments with reference to the accompanying drawings.

In an embodiment of the present invention, a system (10) and a method include an infusion device (100) that comprises of a hollow needle (120) with a pointing end (122) and a shaft (140) that receives the solution for infusion. This solution may include vasopressin, vaso constrictive agent or the like drugs that cannot be directly infused into the blood vessels. These solutions or drugs if directly infused into the blood may results in adverse effect to the body. Solutions like vasopressin is used for constricting blood vessel in order to avoid bleeding and to have clear and bloodless surgeries before a cut is made, for example Laparoscopic surgeries for removal of fibroids also called as myomas from the uterus. This surgery is also known as laparoscopic myomectomy.

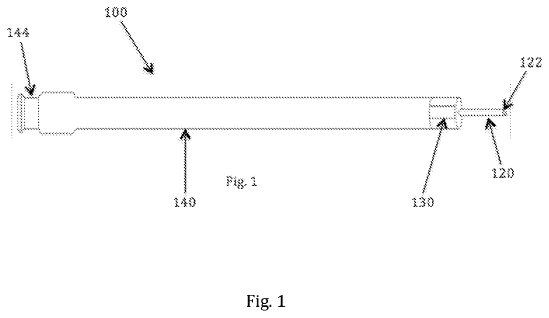

As shown in FIG. 1, the hollow needle (120) of the infusion device has a pointing end (122), which punctures the organ in particular to visceral organs or the blood vessels of the visceral organs. The other end of the hollow needle (120) has a tube (130), which is transparent or translucent or has a transparent surface, which is substantially 1 cm long with a hollow central cylinder of substantially 0.2 cm diameter. The lumen of this tube (130) is continuous with the lumen of the hollow needle (120). The tube (130) on the opposite side is detachably connected with a shaft (140), which is longitudinal hollow and cylindrical to receive solution like vasopressin for infusion into the tissue mass like fibroid. The shaft (140) also includes a hub (144) on the side opposite to the side where the tube (130) is connected. The hub (144) receives the solution like vasopressin from a syringe storing the solution.

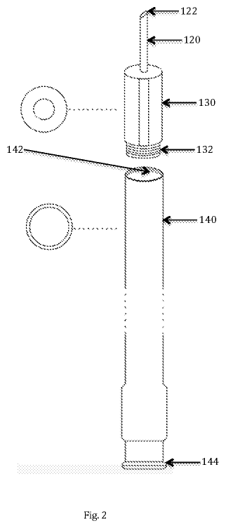

As shown in FIG. 2, the hollow needle (120) of length substantially 2.5 cm has comparatively smaller diameter than the tube (130), which has threads on its outer surface i.e. outer threads (132). The shaft (140) on the other hand has threads on its inner surface i.e. inner threads (142) such that the tube (130) and the shaft (140) can be connected and disconnected as and when required by receiving each other. The shaft (140) may be a metallic shaft that can be reused after sterilization, whereas the hollow needle (120) and the tube (130) may be made of plastic or the like and can be disposed once used. The hollow needle (120) and the tube (130) may be manufactured either as a single piece or as detachable ones.

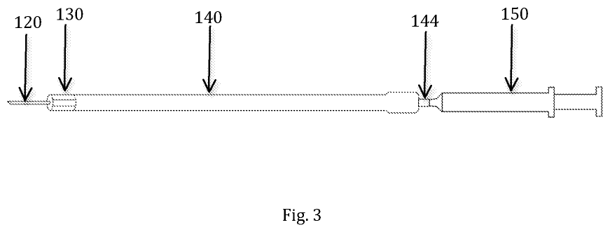

As shown in FIG. 3, the hub (144) of the shaft (140) is structured to receive the syringe (150), which contains the vasopressin. This vasopressin is infused into the fibroid when it is confirmed that the hollow needle (120) has not punctured the blood vessel.

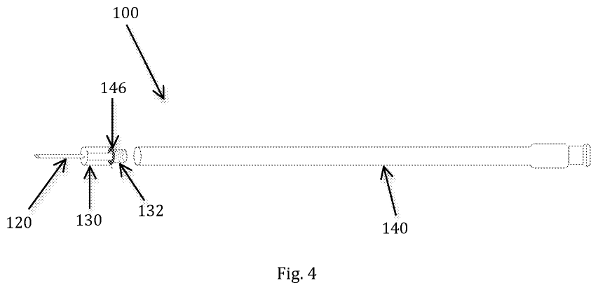

As shown in FIG. 4, the infusion device (100) also includes a seal (146) between the tube (130) and the shaft (140). The seal (146) can be made of rubber or the like material and can have a circular or disc shaped structure with a gap for being received on the outer threads (132), such that when the outer threads (132) receive inner threads (142), the seal functions as a sealing element between the tube (130) and the shaft (140) for preventing solution from leaking when passing through the infusion device (100).

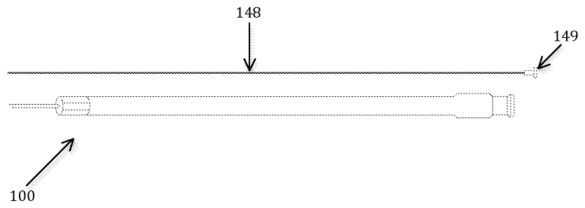

As shown in FIGS. 5 (a) and 5 (b), the infusion device (100) has a length from the hub (144) to the pointing end (122), which is smaller than the length of a stick (148). The stick (148) is a long and thin circular rod shaped structure that can be received by the infusion device (100). The diameter of the stick (148) is lesser than the minimum diameter of the infusion device (100) at any point, so that the stick (148) can be received by the hub (144) of the infusion device (100) and can pass through the hollow needle (120), and form an extension from the pointing end (122), as shown in FIG. 5 (b).

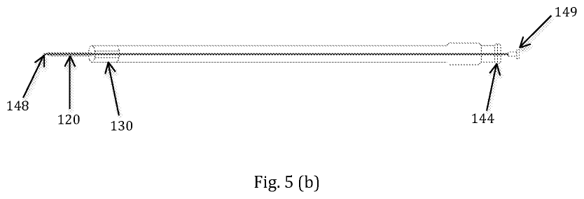

As shown in FIGS. 6 (a) and 6 (b), the system (10) comprises of a duct (170) which is a tube shaped elongated structure to form a first passage having an opening on both sides of the duct such that one opening opens out side the body and the other inside the body. The duct (170) is positioned in an incision or a cut made on the surface of the human body, preferably on or near the abdominal area. The duct after being positioned in the incision makes the first passage for the movement of things like infusion device (100), optical cable etc. to and fro from the interior of the body. The first passage also includes a flap (172) hinged at an inner surface of the duct (170) to block the first passage, thereby avoiding any unnecessary or unintentional movement through the first passage. The flap is structured substantially closer to the opening of the duct (170) facing outside the body, as shown in FIG. 6 (a). The stick (148) has a handle (149) that enables holding of the stick (148). The stick (148) is moved inside the infusion device (100) from the hub (144) and the stick (148) extends or comes out from the pointing end (122). The infusion device (100) therefore forms a second passage for the to and fro movement of the stick (148) for the positioning on the infusion device (100) through and in the duct (170). During the normal condition, when the duct (170) is positioned in the incision or cut on the body surface, the flap (172) covers the first passage being vertical or perpendicular to the longitudinal axis of the first passage of the duct (170). Now, when the infusion device (100) with a stick (148) in inserted into the duct from the opening, the stick (170) being extending outside the pointing end (122) of the hollow needle (120) pushes the flap (172) in the opposite direction, resulting in bending the flap (172) towards the longitudinal direction of the first opening, thereby making or creating a path for the infusion device (100) to pass through the duct (170), as shown in FIG. 6 (b). Since, the stick (148) forms the contact with the flap (172), it prevents pointing end (122) from being damaged by avoiding forming contact with the flap (172). Once the infusion device (100) is passed through the duct (170) i.e. the infusion device (100) is positioned, the stick (148) is withdrawn from the infusion device (100) by pulling it using the handle (149). Thereafter, the hollow needle is positioned continuously until no blood is observed in the tube (130), followed by infusion of solution into the tissue mass or the fibroid.

During laparoscopic myomectomy, before the cut is made on the fibroid, the vasopressin is infused into the fibroid using the infusion device (100), wherein, the infusion device (100) along with the hollow needle (120) is first flushed with dilute vasopressin solution to remove the air column within the infusion device (100). The infusion device (100) is then inserted through any standard 5 mm port into the abdominal cavity. A suitable site over the myoma or fibroid is chosen as per the surgeon's preference and the infusion device (100) is inserted into the fibroid capsule, so as to lie in the sub-capsular plane. Using a 20 ml syringe (150), negative pressure is applied. If the tip of the hollow needle (120) has inadvertently punctured the lumen of a blood vessel over the fibroid i.e. when the pointing end of the needle (120) punctures the fibroid and in case the blood vessel is punctured, the blood from the blood vessel enters the tube (130) through the hollow pathway of the needle (120). If the blood enters into the tube (130) or the cylindrical column within the tube immediately shows a blood stained aspirate, this confirms a positive aspiration test. Alternatively, if the hollow needle (120) is not in a vessel lumen or blood vessel, the aspiration test shows clear fluid i.e. a negative aspiration test. Now, once the blood enters into the tube (130), the surgeon easily observes it and withdraws the hollow needle (120). Once the hollow needle (120) is withdrawn, it is repositioned on the fibroid and in case if the hollow needle (120) again punctures the blood vessel, the blood is observed in the tube (130) i.e. a positive aspiration test is confirmed. The process of repositioning continues until no blood is observed in the tube (130), it confirms that the hollow needle (120) has not punctured any blood vessels of the fibroid i.e. confirming negative aspiration test. Once non-puncturing of the blood vessels of the fibroid is confirmed, the vasopressin stored in the syringe (150) is infused into the fibroid through shaft (140), tube (130) and hollow needle (120). This ensures that the vasopressin has been infused into the fibroids and not directly into the blood vessels.

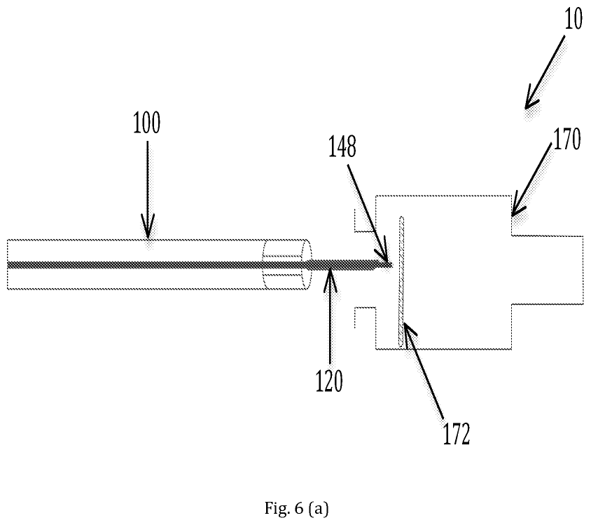

To determine exactly how much volume of blood is required to be aspirated to yield a positive aspiration test, a simple experiment was performed. A container was filled with a dilute solution of methylene blue dye. Three needles were used: a standard 5 mm laparoscopic aspiration needle (160) (case A), a standard 3 mm laparoscopic aspiration needle (162) (case B) and an infusion device (100) of the present invention (case C), as shown in FIG. 7, a 10 ml syringe was connected to each of these 3 needles and aspiration of methylene blue solution was done till blue coloured fluid was seen at the tip of the syringe in (A) and (B), and at the tube of the infusion device in (C). The total quantity of aspirate in each case was collected and measured with a micropipette. It was observed that with a standard 5 mm aspiration needle (160), approximately 0.8 ml of solution was required to demonstrate a positive aspiration test. This quantity was reduced to 0.35 ml with a 3 mm aspiration needle (162), and was remarkably low (0.03 ml) for the infusion device (100). Thus, even an inadvertent puncture of a blood vessel that is of very small calibre can be detected using the infusion device (100), which would quite likely be missed using the other two needles.

### Experiment With and Without the Infusion Device

A total of 53 patients who underwent laparoscopic myomectomy at various hospitals in Mumbai, India were studied over a period of two years. Out of these, 23 patients were operated upon using a standard 5 mm injection needle (Group 1) (160), and 30 patients were operated upon by using the infusion device (100) (Group 2).

A diluted solution of vasopressin (Cpressin P® 20 IU/ml, Samarth Life Sciences, Mumbai, India,) (1 ampoule of 20 units diluted in 200 ml normal saline solution at a dilution of 0.1 U/ml) was used for all cases. The total quantity of diluted vasopressin solution injected ranged from 50 ml to 300 ml, depending on the size of the fibroid, as given below in Table 1.

TABLE 1

| | | Volume of diluted vasopressin solution | |
|---|---|---|---|
| | | used (1 ampoule of 20 units diluted in | |
| Size of Fibroid | | 200 ml NS = 0.1 U/ml) | |
| Less than 4 cm | | 20-50 | ml |
| 4.1 to | 6 cm | 100 | ml |
| 6.1 to | 8 cm | 150 | ml |
| 8.1 to | 10 cm | 200 | ml |
| 10.1 to | 15 cm | 300 | ml |

Each myoma of fibroid was punctured only once at its most prominent portion. Only myomas or fibroid larger than 10 cm were punctured twice, once at most prominent point and once at the base, near it's junction with the normal myometrium. Pulse rate, systolic blood pressure and diastolic blood pressure were measured before injection of vasopressin, at one minute post injection and at 10 minutes post injection. All pulse readings were measured with a finger pulse oximeter probe. All blood pressure (BP) readings were measured with a non invasive blood pressure (NIBP) machine (Philips® SureSignsTM VM8, Koninklijke Philips N.V., Amsterdam, Netherlands) and upper arm BP cuff. Continuous electrocardiography (ECG) monitoring was done for all patients. A post-injection elevation of pulse or blood pressure of 20 percent over the pre injection value was considered significant. The following details were recorded for each patient:
1. Type of Needle used: Regular/Infusion device
2. Number of fibroids removed
3. Size of largest fibroid
4. Pre injection Pulse
5. Pre injection blood pressure
6. Pulse and Blood Pressure at 1 min after injection
7. Pulse and Blood Pressure at 10 min after injection
8. Any ECG changes suggesting myocardial ischemia/arrhythmia
9. If Infusion device used: was blood seen in tube?

### Observation

Out of the 30 patients in whom the infusion device (100) was used, 4 positive aspirations (i.e. blood seen at the infusion device's tube) were found (13.33%). Once a positive aspiration was noted, the infusion device was withdrawn and re-inserted at a new location. An aspiration test was repeated, and the vasopressor solution was injected only after confirming a negative aspiration test. None of the 30 patients in whom infusion device was used after confirming a negative aspiration demonstrated any significant change in post-injection pulse or blood pressure recordings.

Out of the 23 patients in whom a regular needle (160) was used, and aspiration showed no blood within the syringe, 4 patients (17.39%) demonstrated a significant (over 20 percent of pre injection value) but transient elevation in pulse and blood pressure readings at 1 minute post injection. This gradually returned to baseline at 10 minutes after the injection. None of the patients had any ischaemic ECG changes or significant adverse cardiovascular events.

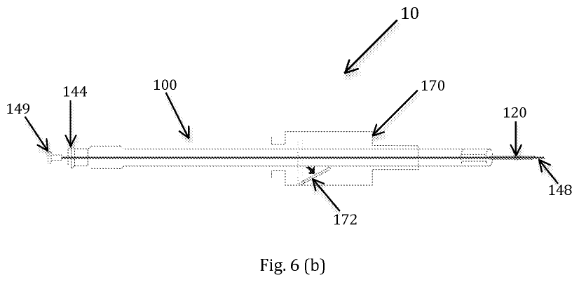

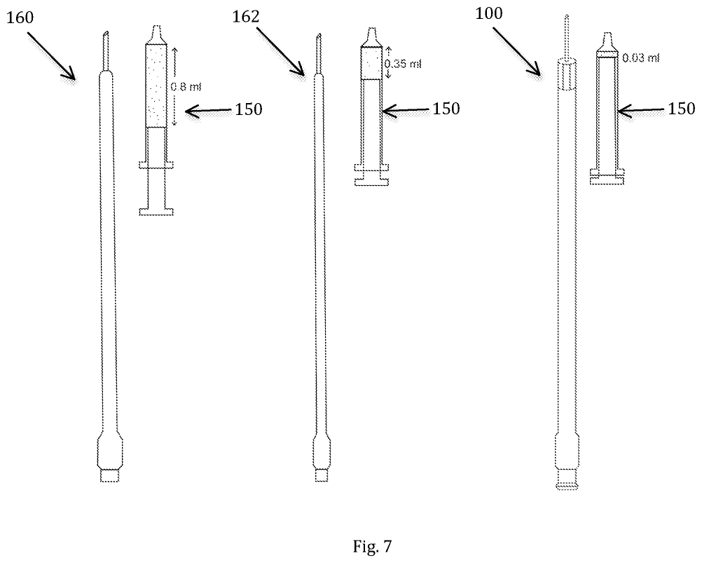

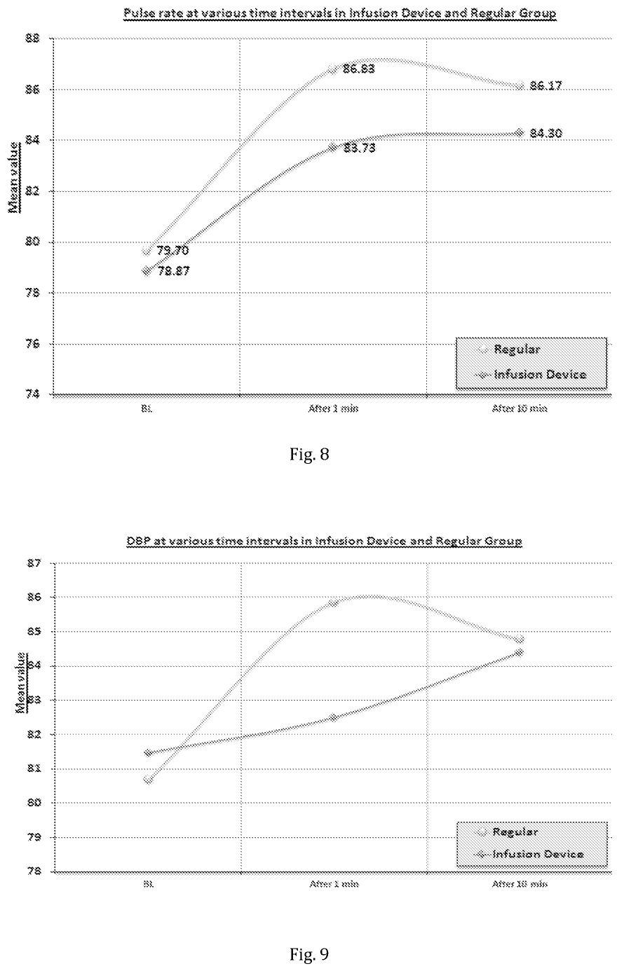

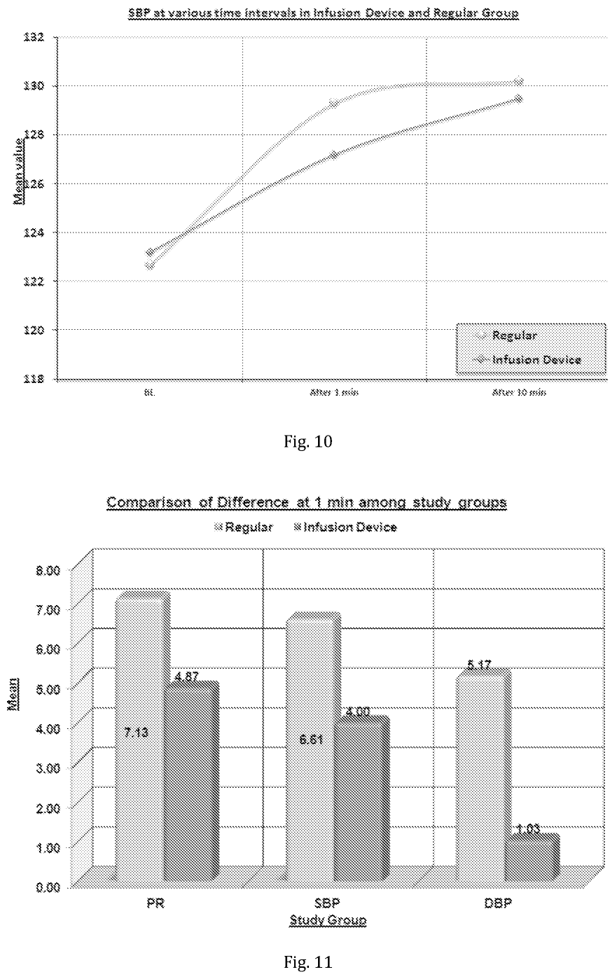

As shown in FIGS. 8 and 9, the mean elevation in pulse rate and systolic blood pressure at 1 minute was higher in Group 1 (Regular needle group) than Group 2 (Infusion device group) as shown in below Table 2. As shown in FIG. 10, the mean diastolic blood pressure showed a statistically significant higher elevation at 1 minute in Group 1 rather than Group 2. As shown in FIG. 11, the mean of pulse rate, systolic blood pressure and diastolic blood pressure with regular needle is comparatively very high than respective mean of pulse rate, systolic blood pressure and diastolic blood pressure with infusion device.

TABLE 2

| | Regular (Number | Infusion Device (Number |
|---|---|---|
| Pulse (Mean) | of people = 23) | of people = 30) |
| Baseline | 79.70 | 78.87 |
| After 1 min | 86.83 | 83.73 |
| After 10 min | 86.17 | 84.30 |
| Systolic Blood Pressure | | |
| (Mean) | Regular | Infusion Device |
| Baseline | 122.70 | 123.20 |
| After 1 min | 129.30 | 127.20 |
| After 10 min | 130.22 | 129.50 |
| Diastolic Blood Pressure | | |
| (Mean) | Regular | Infusion Device |
| Baseline | 80.70 | 81.47 |
| After 1 min | 85.87 | 82.50 |
| After 10 min | 84.78 | 84.40 |

Therefore, the use of infusion device as in present invention provides positive aspiration test as the puncturing of blood vessel is observed and confirmed the infusion of vasopressin in the fibroid and not directly into the blood vessel, thereby avoiding any cardiovascular complications.

The present invention is however not limited to the above embodiments disclosed above and referred in FIGS. 1 to 11 and other embodiments within the scope of the invention can be used for achieving the result of the present invention without limiting the scope of the invention.

1. An infusion device comprising:
a hollow needle having a pointing end for puncturing a tissue mass of a visceral organ;
a tube at an opposite side of said pointing end of said hollow needle for observing blood in said tube when said pointing end of said hollow needle punctures a blood vessel of said visceral organ; and
a longitudinal hollow cylindrical shaft connected to said structured to receive a solution for infusion into said tissue mass,
wherein said needle is withdrawn from said blood vessel and repositioned on said visceral organ when blood is observed in said tube, and said solution is infused into said tissue mass of said visceral organ when no blood is observed in said tube after repositioning of said needle on said organ, thereby ensuring infusion of said solution into said tissue mass.

2. The infusion device as claimed in claim 1, wherein said tissue mass is a fibroid.

3. The infusion device as claimed in claim 1, wherein said tube is transparent to observe blood.

4. The infusion device as claimed in claim 1, wherein said shaft is detachably connected to said tube.

5. The infusion device as claimed in claim 4, wherein said shaft and said tube are threaded to receive each other.

6. The infusion device as claimed in claim 5, wherein said shaft is a metallic shaft.

7. The infusion device as claimed in claim 6, wherein said shaft includes a hub to receive a syringe carrying said solution for infusion into said tissue mass.

8. The infusion device as claimed in claim 1, wherein said tube is substantially 1 cm long with a hollow central cylinder of substantially 0.2 cm diameter.

9. The infusion device as claimed in claim 1, wherein said hollow needle is substantially 2.5 cm long.

10. The infusion device as claimed in claim 5, wherein shaft and said tube receive each other with a seal in between said shaft and said tube.

11. A method for infusion of a solution into a tissue mass of a visceral organ, including the steps of:
puncturing said visceral organ by a pointing end of a hollow needle having a tube on an opposite side of said hollow needle;
observing blood in said tube of said hollow needle when said pointing end punctures a blood vessel of said visceral organ;
withdrawing and repositioning said hollow needle when blood is observed in said tube of said hollow needle; and
infusing said solution into said tissue mass without directly infusing into said blood vessel by a longitudinal hollow cylindrical shaft connected to said tube when blood is not observed in said tube after puncturing said tissue mass by said hollow needle.

12. The method as claimed in claim 11, including the step of infusing said solution into said tissue mass by infusing said solution into a fibroid.

13. The method as claimed in claim 11, including the step of observing blood by said tube with transparent surface.

14. The method as claimed in claim 11, including the step of infusing said solution into said tissue mass by detachably connecting said shaft with said tube of said hollow needle.

15. The method as claimed in claim 14, including the step of infusing said solution into said tissue mass by said shaft receiving said tube with both having threads.

16. The method as claimed in claim 13, including the step of infusing said solution into said tissue mass by a syringe having said solution and said syringe is received by a hub of said shaft on opposite side of said shaft connected with said tube.

17. The method as claimed in claim 11, including the step of observing blood in said tube substantially 1 cm long with a hollow central cylinder of substantially 0.2 cm diameter.

18. The method as claimed in claim 17, including the step of puncturing said tissue mass by said pointing end of said hollow needle substantially 2.5 cm long.

19. A system for infusing a solution into a tissue mass comprising:
a duct with an opening on both side positioned in an incision on a body surface for forming a first passage including a flap at an inner surface of said duct in said first passage;
a stick for pushing said flap to open said first passage; and an infusion device of length smaller than length of said stick forming a second passage through said infusion device to allow said stick to pass through said infusion device, said infusion device includes a hollow needle having a pointing end for puncturing said tissue mass of a visceral organ, a tube at an opposite side of said pointing end of said hollow needle for observing blood in said tube when said pointing end of said hollow needle punctures a blood vessel of said visceral organ, and a longitudinal hollow cylindrical shaft connected to said tube structured to receive a solution for infusion into said tissue mass,
wherein said stick passes through said hollow needle of said infusion device when received in said infusion device for positioning said infusion device through said duct by pushing said flap by said stick, thereafter said stick is withdrawn from said infusion device, and said needle is withdrawn from said blood vessel and repositioned on said visceral organ when blood is observed in said tube, and said solution is infused into said tissue mass of said visceral organ when no blood is observed in said tube after repositioning of said needle on said visceral organ, thereby ensuring infusion of said solution into said tissue mass.

20. The system as claimed in claim 19, wherein said tissue mass is a fibroid.

21. The system as claimed in claim 19, wherein said tube is transparent to observe blood.

22. The system as claimed in claim 19, wherein said shaft is detachably connected to said tube.

23. The system as claimed in claim 22, wherein said shaft and said tube are threaded to receive each other.

24. The infusion device as claimed in claim 23, wherein said shaft and said tube receive each other with a seal in between said shaft and said tube.

25. The system as claimed in claim 22, wherein said shaft includes a hub to receive a syringe carrying said solution for infusion into said tissue mass after withdrawing said stick from said infusion device.

26. The system as claimed in claim 23, wherein said tube is substantially 1 cm long with a hollow central cylinder of substantially 0.2 cm diameter.

27. The system as claimed in claim 19, wherein said hollow needle is substantially 2.5 cm long.

28. A method for infusion of a solution into a tissue mass of a visceral organ, including the steps of:
positioning a duct with an opening on both side in an incision on a body surface for forming a first passage including a flap at an inner surface of said duct in said first passage;
receiving a stick by an infusion device of length smaller than length of said stick forming a second passage through said infusion device to allow said stick to pass through said infusion device;
positioning said infusion device through said duct by pushing said flap by said stick received by said infusion device and passing through a hollow needle of said infusion device;
withdrawing said stick from said infusion device after positioning said infusion device through said duct;
puncturing said visceral organ by a pointing end of said hollow needle having a tube on an opposite side of said hollow needle; observing blood in said tube of said hollow needle when said pointing end punctures a blood vessel of said visceral organ;
withdrawing and repositioning said hollow needle when blood is observed in said tube of said hollow needle; and
infusing said solution into said tissue mass without directly infusing into said blood vessel by a longitudinal hollow cylindrical shaft connected to said tube when blood is not observed in said tube after puncturing said tissue mass by said hollow needle.

29. The method as claimed in claim 28, including the step of infusing said solution into said tissue mass by infusing said solution into a fibroid.

30. The method as claimed in claim 28, including the step of observing blood by said tube with a transparent surface.

31. The method as claimed in claim 28, including the step of infusing said solution into said tissue mass by detachably connecting said shaft with said tube of said hollow needle.

32. The method as claimed in claim 31, including the step of infusing said solution into said tissue mass by said shaft receiving said tube with both having threads.

33. The method as claimed in claim 31, including the step of infusing said solution into said tissue mass by a syringe having said solution and said syringe is received by a hub of said shaft on opposite side of said shaft connected with said tube.

34. The method as claimed in claim 28, including the step of observing blood in said tube substantially 1 cm long with a hollow central cylinder of substantially 0.2 cm diameter.

35. The method as claimed in claim 28, including the step of puncturing said tissue mass by said pointing end of said hollow needle substantially 2.5 cm long.
